(19) United States
(12) Patent Application Publication
(10) Pub. No.: US 2006/0079579 A1
(43) Pub. Date: Apr. 13, 2006

(54) Combinations of valsartan with cox-2 inhibitors

(76) Inventors: Francis Plat, Riedisheim (FR); Giles Victor Campion, Oxford (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/537,869

(22) PCT Filed: Dec. 17, 2003
(86) PCT No.: PCT/EP03/14406

§ 371 Date: Jul. 6, 2005

Related U.S. Application Data
(60) Provisional application No. 60/434,250, filed on Dec. 18, 2002.

(30) Foreign Application Priority Data
Dec. 18, 2002 (US) 60434250

(51) Int. Cl. A61K 31/195

(52) U.S. Cl. 514/567

(57) ABSTRACT

The invention relates a pharmaceutical composition comprising a combination of (i) the AT 1-receptor blocker valsartan or a pharmaceutically acceptable salt thereof and (ii) a COX-2 inhibitor or a pharmaceutically acceptable salt thereof and optionally a pharmaceutically acceptable carrier and to a method for the treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, comprising administering a therapeutically effective amount of the pharmaceutical composition to a mammal in need thereof.

The present invention relates to pharmaceutical compositions comprising the AT1 receptor blocker valsartan or pharmaceutically acceptable salts thereof and a COX-2 inhibitor or a pharmaceutically effective salts thereof, optionally in the presence of a pharmaceutically acceptable carrier.

The present invention furthermore relates to pharmaceutical compositions which comprise in combination the AT 1 -receptor blocker valsartan and a COX-2 inhibitor selected from the group of: compound of formula V 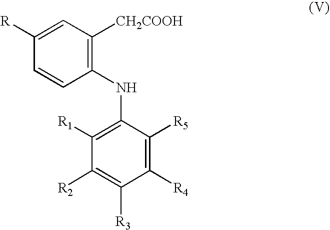
wherein R is methyl or ethyl;
R1 is chloro or fluoro;
R2 is hydrogen or fluoro;
R3 is hydrogen, fluoro, chloro, methyl, ethyl, methoxy, ethoxy or hydroxy;
R4 is hydrogen or fluoro; and
R5 is chloro, fluoro, trifluoromethyl or methyl;
pharmaceutically acceptable salts thereof; and
pharmaceutically acceptable prodrug esters thereof
   or a pharmaceutically acceptable salt thereof and a pharmaceutically acceptable carrier for simultaneous, sequential or separate use.

Valsartan is the AT 1-receptor blocker (S)-N-(1-carboxy-2-methyl-prop-1-yl)-N-pentanoyl-N-[2;(1H-tetrazol-5-yl)biphenyl-4-yl-methyl]amine of formula (I) 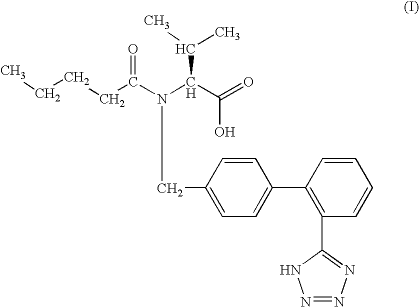
and is disclosed in EP 0443983 A and U.S. Pat. No. 5,399,578, the disclosures of which are incorporated herein in their entirety as if set forth herein.

Particularly preferred compounds of formula V are those wherein R is methyl or ethyl; R1 is chloro or fluoro; R2 is hydrogen; R3 is hydrogen, fluoro, chloro, methyl or hydroxy; R4 is hydrogen; and R5 is chloro, fluoro or methyl; pharmaceutically acceptable salts thereof; and pharmaceutically acceptable esters thereof.

A particularly preferred embodiment relates to the compounds of formula V wherein R is methyl or ethyl; R1 is fluoro; R2 is hydrogen; R3 is hydrogen, fluoro or hydroxy; R4 is hydrogen; and R5 is chloro; pharmaceutically acceptable salts thereof; and pharmaceutically acceptable prodrug esters thereof.

Another particularly preferred embodiment of the invention relates to compounds of formula V wherein R is ethyl or methyl; R1 is fluoro; R2 is hydrogen or fluoro; R3 is hydrogen, fluoro, ethoxy or hydroxy; R4 is hydrogen or fluoro; and R5 is chloro, fluoro or methyl; pharmaceutically acceptable salts thereof; and pharmaceutically acceptable prodrug esters thereof.

Further are said compounds wherein R is methyl or ethyl; R1 is fluoro; R2-R4 are hydrogen or fluoro; and R5 is chloro or fluoro; pharmaceutically acceptable salts thereof; and pharmaceutically acceptable prodrug esters thereof.

A further embodiment of the invention relates to the compounds of formula V wherein R is methyl or ethyl; R1 is fluoro; R2 is fluoro; R3 is hydrogen, ethoxy or hydroxy; R4 is fluoro; and R5 is fluoro; pharmaceutically acceptable salts thereof; and pharmaceutically acceptable prodrug esters thereof.

Another embodiment of the invention relates to the compounds of formula V wherein R is methyl; R1 is fluoro; R2 is hydrogen; R3 is hydrogen or fluoro; R4 is hydrogen; and R5 is chloro; pharmaceutically acceptable salts thereof; and pharmaceutically acceptable prodrug esters thereof.

Particularly preferred embodiments of the invention relate to compounds of formula V

(a) wherein R is methyl; R1 is fluoro; R2 is hydrogen; R3 is hydrogen; R4 is hydrogen; and R5 is chloro; pharmaceutically acceptable salts thereof; and pharmaceutically acceptable prodrug esters thereof;

(b) wherein R is methyl; R1 is fluoro; R2 is hydrogen; R3 is fluoro; R4 is hydrogen; and R5 is chloro; pharmaceutically acceptable salts thereof; and pharmaceutically acceptable prodrug esters thereof;

(c) wherein R is ethyl; R1 is fluoro; R2 is fluoro; R3 is hydrogen; R4 is fluoro; and R5 is fluoro; pharmaceutically acceptable salts thereof; and pharmaceutically acceptable prodrug esters thereof; and

wherein R is ethyl; R1 is chloro; R2 is hydrogen; R3 is chloro; R4 is hydrogen; and R5 is methyl; pharmaceutically acceptable salts thereof; and pharmaceutically acceptable prodrug esters thereof.

Pharmaceutically acceptable prodrug esters of the compounds of formula V are ester derivatives which are convertible by solvolysis or under physiological conditions to the free carboxylic acids of formula V. Such esters are e.g. lower alkyl esters (such as the methyl or ethyl ester), carboxy-lower alkyl esters such as the carboxymethyl ester, nitrooxy-lower alkyl esters (such as the 4-nitrooxybutyl ester), and the like. Preferred prodrugs are the compounds of formula Ia 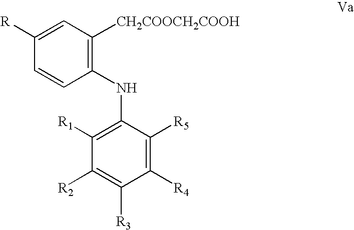
wherein R and R1-R5 have meaning as defined hereinabove for compounds of formula V; and pharmaceutically acceptable salts thereof.

Compounds of formula V and Va and their synthesis are described in published international patent applications Nos. WO 99/11605 and WO 01/23346, the teachings of which are incorporated herein by reference, especially the corresponding compounds generically and specifically disclosed in the examples and claims.

Most preferred COX-2 inhibitor of formula V to be used in the present invention are

CGS35189: 5-methyl-2-(2′-chloro -6′fluororanilino)phenylacetic acid of formula 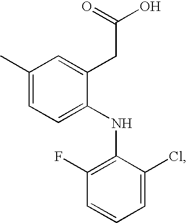
CGS35944: 5-methyl-2(2′,4′-difluoro-6′-chloroanilino)phenylacetic acid of formula 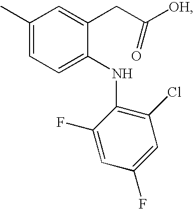
and CGS34975: 5-ethyl-2-(2′,3′,5′,6′-tetrafluoroanilino)phenylacetic acid of formula 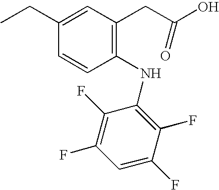
or a pharmaceutically acceptable salts thereof.

An alternative class of cox-2 inhibitors compounds for use in the invention is the methane sulfonanilide class of inhibitors, of which NS-398, flosulide, nimesulide and (i) are example members.

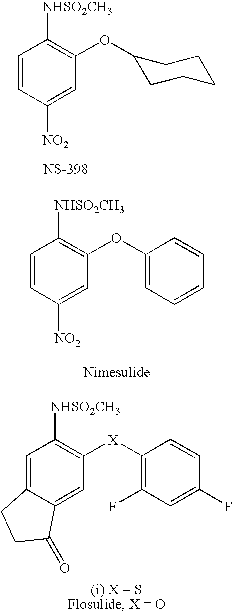

A further class of COX-2 inhibitors is the tricyclic inhibitor class, which can be further divided into the sub-classes of tricyclic inhibitors with a central carbocyclic ring (examples include SC-57666, 1 and 2; those with a central monocyclic heterocyclic ring (examples include DuP 697, SC-58125, SC-58635, SC 236 and 3,4 and 5); and those with a central bicyclic heterocyclic ring (examples include 6, 7, 8, 9 and 10). Compounds 3, 4, and 5 are described in U.S. Pat. No. 5,474,995.

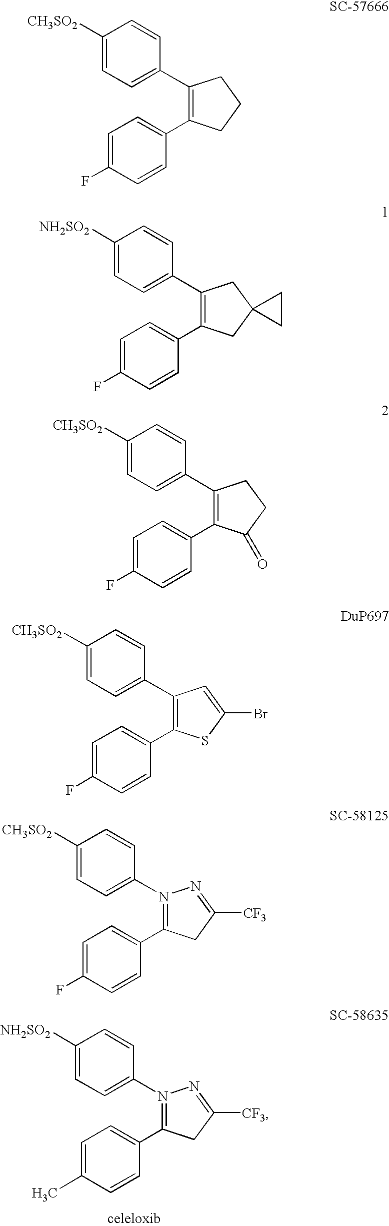

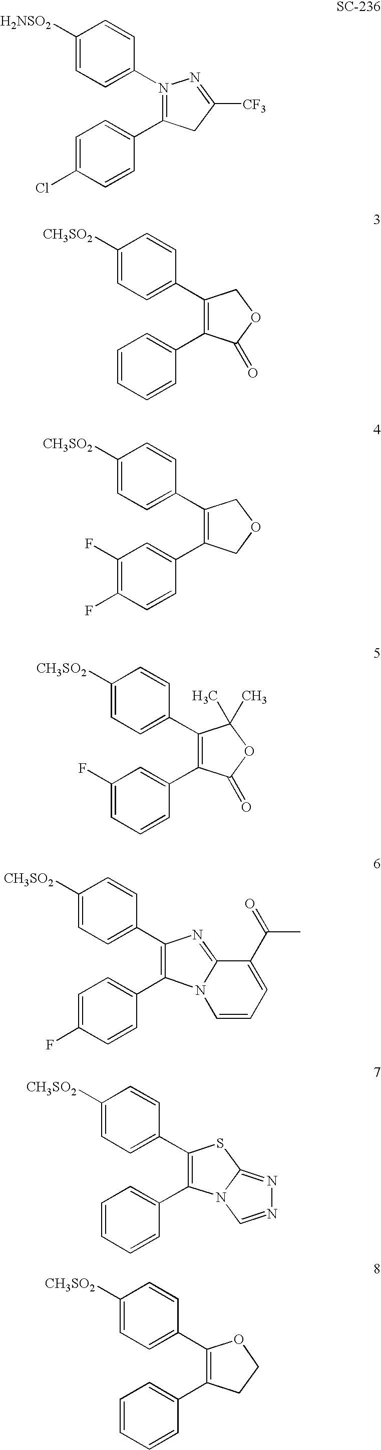

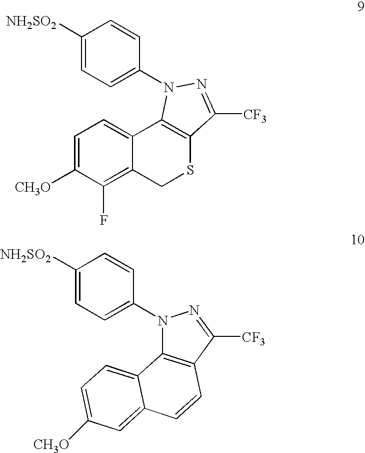

A yet further class of COX-2 inhibitors can be referred to as those which are structurally modified NSAIDS, and includes 11a and structure 11 as example members.

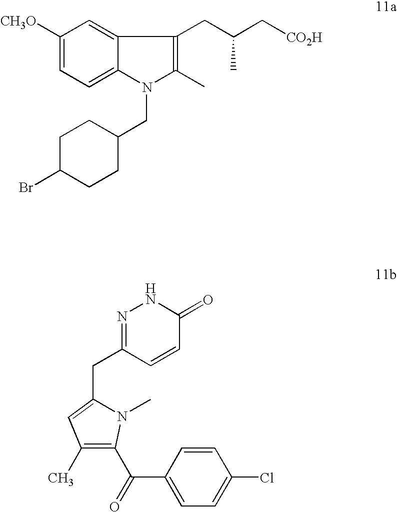

In addition to the structural classes, sub-classes, specific COX-2 inhibitors compound examples, examples of compounds which selectively inhibit cyclooxygenase-2 have also been described in the following patent publications, all of which are herein incorporated by reference: U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780; and International Patent Specification Nos. 94/13635, 94/15932, 94/20480, 94/26731, 94/27980, 95/00501, 95/15316, 96/03387, 96/03388, 96/06840; and International Publication No.'s WO 94/20480, WO 96/21667, WO 96/31509, WO 96/36623, WO 97/14691, WO 97/16435.

Additional COX-2 inhibitor compounds which are included in the scope of this invention include:

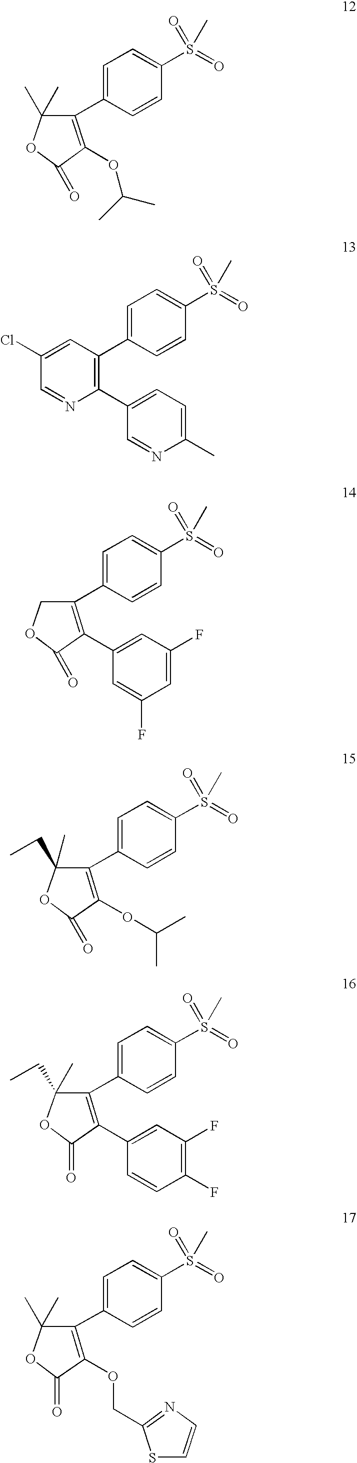

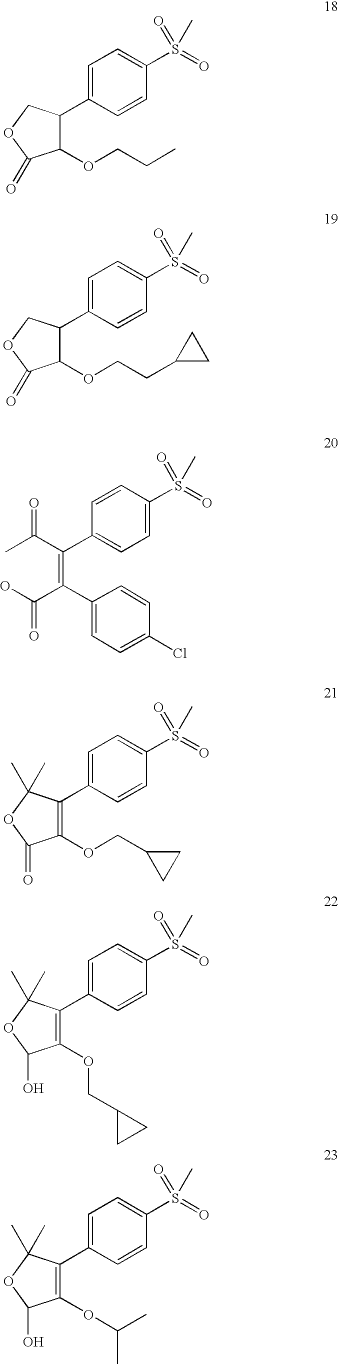

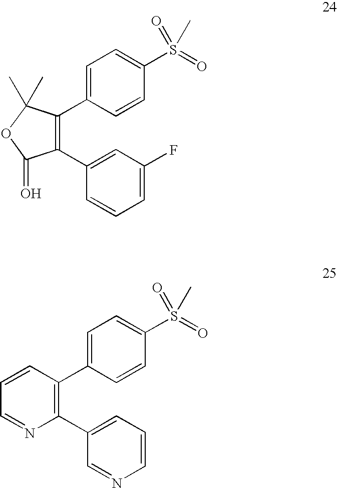

Some of the compounds above can also be identified by the following chemical names:

3: 3-phenyl-4-(4-(methylsulfonyl)phenyl)-2-(5H)-furanone;

4: 3-(3,4-difluorophenyl)-4-(4-(methylsulfonyl)phenyl)-2-(5H)-faranone;

5: 5,5-dimethyl-4-(4-(methylsulfonyl)phenyl)-3-(3-fluorophenyl)-H-furan-2-one;

12: 5,5-dimethyl-4-(4-(methylsulfonyl)phenyl)-3-(2-propoxy)-5H-furan-2-one;

13: 5-chloro-3-(4-(methylsulfonyl)phenyl)-2-(2-methyl-5-pyridinyl)pyridine;

14:2-(3,5-difluorophenyl)-3-(4-(methylsulfonyl)phenyl-2-cyclopenten-1-one;

15: 5(S)-5-ethyl-5-methyl-4-(4-methylsulfonyl)phenyl)-3-(2-propoxy)-5H-furan-2-one;

16: 5-ethyl-5-methyl-4-(4-(methylsulfonyl)phenyl)-3-(3,4-difluorophenyl)-5H-furan-2-one;

17: 3-((2-thiazolyl)methoxy)-4-(4-methylsulfonyl)phenyl)-5,5-dymethyl-5H-furan-2-one;

18: 3-propyloxy-4-(4-methylsulfonyl)phenyl)-5,5-dimethyl-5H-furan-2-one;

19: 3-(1-cyclopropylethoxy)-5,5-dimethyl-4-(4-methylsulfonyl)phenyl)-5H-furan-2-one;

20: sodium 2-(4-chlorophenyl)-3-(4-methylsulfonyl)phenyl)-4-oxo-2-pentenoate;

21: 3-(cyclopropylmethoxy)-5,5-dimethyl-4-(4-methylsulfonyl)phenyl)-5H-furan-2-one;

22: 3-(cyclopropylmethoxy)-5,5-dimethyl-4-(4-methylsulfonyl)phenyl)-2,5-dihydrofuran-2-ol;

23:3-isopropoxy-5,5-dimethyl-4-(4-methylsulfonyl)phenyl)-2,5-dihydrofuran-2-ol;

24: 5,5-dimethyl-3-(3-fluorophenyl)-2-hydroxy-4-(4-methylsulfonyl)phenyl)-2,5-dihydrofuran;

25: 5-Chloro-3-(4-methylsulfonyl)phenyl)-2-(3-pyridinyl)pyridine.

The following publications describe and/or provide methods for making the compounds as indicated: compounds 12, 15, 17, 18, 19 and 21, WO 97/14691; compounds 22,23 and 24, WO 97/16435; compound 20, WO 96/36623; compound 14, U.S. Pat. No. 5,536,752; compound 16, U.S. Pat. No. 5,474,995. See Examples herein for compounds 13 and 25.

Also incorporated herein by reference are those compounds described in WO 96/41645 as having structural Formula VI, shown below, and the definition and preferred definitions and species described therein:

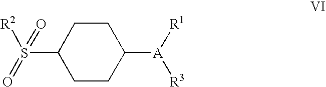

Particulary preferred compounds of formula (VI) include:

5-(4-fluorophenyl)-1-[4-(methylsulfonyl)phenyl]-3-(trifluoromethyl)pyrazole;

4-(4-fluorophenyl)-5-[4-(methylsulfonyl)phenyl]-1-phenyl-3-(trifluoromethyl)pyrazole;

4-(5-(4-chlorophenyl)-3-(4-methodoxyphenyl)-1H-pyrazol-1-yl)benzenesulfonamide;

4-(3,5-bis(4-methylphenyl)-1H-pyrazol-1-yl)benzenesulfonamide;

4-(5-(4-chlorophenyl)-3-phenyl-1H-pyrazol-1-yl)benzenesulfonamide;

4-(3,5-bis(4-methoxyphenyl)-1H-pyrazol-1-yl)benzenesulfonamide;

4-(5-(4-chlorophenyl)-3-(4-methylphenyl)-1H-pyrazol-1-yl)benzenesulfonamide;

4-(5-(4-chlorophenyl)-3-(4-nitrophenyl)-1H-pyrazol-1-yl)benzenesulfonamide;

4-(5-(4-chlorophenyl)-3-(5-chloro-2-thienyl)-1H-pyrazol-1-yl)benzenesulfonamide;

4-(4-chloro-3,5-diphenyl-1H-pyrazol-1-yl)benzenesulfonamide;

4-(5-(4-chlorophenyl)-3-(trifluoromethyl)-1H-pyrazol-1-yl)benzenesulfonamide;

4-(5-phenyl)-3-(trifluormethyl)-1H-pyrazol-1-yl)benzenesulfonamide;

4-(5-(4-fluorphenyl)-3-(trifluoromethyl)-1H-pyrazol-1-yl)benzenesulfonamide;

4-(5-(4-methoxyphenyl)-3-(trifluormethyl)-1H-pyrazol-1-yl)benzenesulfonamide;

4-(5-(4-chlorophenyl)-3-(difluoromethyl)-1H-pyrazol-1-yl)benzenesulfonamide;

4-(5-(4-methylphenyl)-3-(trifluoromethy)-1H-pyrazol-1-yl)benzenesulfonamide;

4-(4-chloro-5-(4-chlorohenyl)-3-(trifluoromethyl)-1H-pyrazol-1-yl)benzenesulfonamide;

4-(3-(difluoromethyl)-5-(4-methylphenyl)-1H-pyrazol-1-yl)benzenesulfonamide;

4-(3-(difluoromethyl)-5-phenyl-1H-pyrazol-1-yl)benzenesulfonamide;

4-(3-(difluoromethyl)-5-(4-methoxyphenyl)-1H-pyrazol-1-yl)benzenesulfonamide;

4-(3-cyano-5-(4-fluorophenyl)-1H-pyrazol-1-yl)benzenesulfonamide;

4-(3-(difluoromethyl)-5-(3-fluoro-4-methodoxyphenyl)-1H-pyrazol-1-yl)benzenesulfonamide;

4-(5-(3-fluoro-4-methoxyphenyl)-3-(trifluormethyl)-1H-pyrazol-1-yl)benzenesulfonamide;

4-(4-chloro-5-phenyl-1H-pyrazol-1-yl)benzenesulfonamide;

4-(5-(4-chlorophenyl)-3-hydroxyphenyl)-1H-pyrazol-1-yl)benzenesulfonamide;

4-(5-(N,N-dimethylamino)phenyl)-3-(trifuoromethyl)-1H-pyrazol-1-yl)benzenesulfonamide;

5-(4-fluorophenyl)-6-(4-(methylsulfonyl)phenyl)spiro[2.4]hept-5-ene;

4-(6-(4-fluorophenyl)spiro[2.4]hept-5-en-5yl)benzenesulfonamide;

6-(4-fluorophenyl)-7-(4-(methylsulfonyl)phenyl)spiro[3.4]oct-6-ene;

5-(3-chloro-4-methoxyphenyl)-6-(4-(methylsulfonyl)phenyl)spiro[2.4]hept-5-ene;

4-(6-(3-chloro-4methoxyphenyl)spiro[2.4]hept-5-en-5-yl)benzenesulfonamide;

5-(3,5-dichloro-4-methodoxyphenyl)-6-(4-(methylsulfonyl)phenyl)spiro[2.4]hept-5-ene;

5-(3-chloro-4-fluorophenyl)-6-(4-(methylsulfonyl)phenyl)spiro[2.4]hept-5-ene;

4-(6-(3,4-dichlorophenyl)spiro[2.4]hept-5-en-5-yl)benzenesulfonamide;

2-(3-chloro-4-fluorophenyl)-4-(4-fluorophenyl)-5-(4-methylsulfonylphenyl)thiazole;

2-(2-chlorophenyl)-4-(4-fluorophenyl)-5-(4-methylsulfonylphenyl)thiazole;

5-(4-fluorophenyl)-4-(4-methylsulfonylphenyl)-2-methylthiazole;

4-(4-fluorophenyl)-5-(4-methylsulfonylphenyl)-2-trifluormethylthiazole;

4-(4-fluorophenyl)-5-(4-methylsulfonylphenyl)-2-(2-thienyl)thiazole;

4-(4-fluorophenyl)-5-(4-methylsulfonylphenyl)-2-benzenesulfonamide;

4-(4-fluorophenyl)-5-(4-methylsulfonylphenyl)-2-(1-propylamino)thiazole;

2-((3,5-dichlorophenoxy)methyl)-4-(4-fluorophenyl)-5-(4-(methylsulfonyl)phenyl)thiazole;

5-(4-fluorophenyl)-4-(4-methylsulfonylphenyl)-2-trifluoromethylthiazole;

1-methylsulfonyl-4-(1,1-dimethyl-4-(4-fluorophenyl)cyclopenta-2,4-dien-3-yl)benzene;

4-(4-(4-fluorophenyl-1,1-dimethylcyclopenta-2,4-dien-3-yl)benzenesulfonamide;

5-(4-fluorophenyl)-6-(4-(methylsulfonyl)phenyl)spiro[2.4]hepta-4,6-diene;

4-(6-(4-fluorophenyl)spiro[2.4]hepta-4,6-dien-5-yl)benzenesulfonamide;

6-(4-fluorophenyl)-2-methoxy-5-(4-(methylsulfonyl)phenyl)-pyridine-3-carbonitrile;

2-bromo-6-(4-fluorophenyl)-5-(4-(methylsulfonyl)phenyl)-pyridine-3-carbonitrile;

6-(4-fluorophenyl)-5-(4-(methylsulfonyl)phenyl)-2-phenyl-pyridine-3-carbonitrile;

4-(2-(4-methylpyridin-2-yl)-4-(trifluoromethyl)-1H-imidazol-1-yl)benzenesulfonamide;

4-(2-(5-methylpyridin-3-yl)-4-(trifluoromethyl)-1H-imidazol-1-yl)benzenesulfonamide;

4-(2-(2-methylpyridin-3-yl)-4-(trifluoromethyl)-1H-imidazol-1-yl)benzenesulfonamide;

3-(1-(4-(methylsulfonyl)phenyl)-4-(trifluoromethyl)-1H-imidazol-2-yl)benzenesulfonamide;

2-(1-(4-(methylsulfonyl)phenyl)-4-(trifluoromethyl)-1H-imidazol-2-yl)pyridine;

2-methyl-4-(1-(4-(methylsulfonyl)phenyl)-4-(trifluormethyl)-1H-imidazol-2-yl)pyridine;

2-methyl-6-(1-(4-(methylsulfonyl)phenyl)-4-(trifluormethyl)-1H-imidazole-2-yl)pyridine;

4-(2-(6-methylpyridin-3-yl)-4-(trifluoromethyl)-1H-imidazol-1-yl)benzenesulfonamide;

2-(3,4-difluorophenyl)-1-(4-(methylsulfonyl)phenyl)-4-(trifluoromethyl)-1H-imidazole;

4-(2-(4-methylphenyl)-4-(trifluoromethyl)-1H-imidazol-1-yl)benzesulfonamide;

2-(4-chlorophenyl)-1-(4-methylsulfonyl)phenyl)-4-methyl-1H-imidazole;

2-(4-chlorophenyl)-1-(4-(methylsulfonyl)phenyl)-4-phenyl-1H-imidazole;

2-(4-chlorophenyl)-4-(4-fluorophenyl)-1-(4-(methylsulfonyl)phenyl)-1H-imidazole;

2-(3-fluoro-4-methoxyphenyl)-1-(4-(methylsulfonyl)phenyl)-4-(trifluoromethyl)-1H-imidazole;

1-(4-methylsulfonyl)phenyl)-2-phenyl-4-trifluoromethyl-1H-imidazole;

2-(4-methylphenyl)-1-(4-(methylsulfonyl)phenyl)-4-trifluoromethyl-1H-imidazole;

4-(2-(3-chloro-4-methylphenyl)-4-(trifluoromethyl-1H-imidazol-1-yl)benzenesulfonamide;

2-(3-fluoro-5-methylphenyl)-1-(4-methylsulfonyl)phenyl)-4-(trifluoromethyl)-1H-imidazole;

4-(2-(3-fluoro-5-methylphenyl)-4-(trifluoromethyl)-1H-imidazol-1-yl)benzenesulfonamide;

2-(3-methylphenyl)-1-(4-(methylsulfonyl)phenyl)-4-(trifluoromethyl)-1H-imidazole;

4-(2-(3-methylphenyl)-4-(trifluoromethyl)1H-imidazol-1-yl)benzenesulfonamide;

1-(4-(methylsulfonyl)phenyl)-2-(3-chlorophenyl)-4-(trifluoromethyl)-1H-imidazole;

4-(2-(3-chlorophenyl)-4-(trifluoromethyl)-1H-imidazol-1-yl)benzenesulfonamide;

4-(2-phenyl-4-(trifuoromethyl)-1H-imidazol-1-yl)benzenesulfonamide;

4-(2-(4-methodxy-3-chlorophenyl)-4-(trifluoromethyl)-1H-imidazol-1-yl)benzenesulfonamide;

1-allyl-4-(4-fluorophenyl)-3-(4-(methylsulfonyl)phenyl)-5-(trifluoromethyl)-1H-pyrazole;

4-(1-ethyl-4-(4-fluorophenyl)-5-(trifluoromethyl)-1H-pyrazol-3-yl)benzenesulfonamide;

N-phenyl-(4-(4-fluorophenyl)-3-(4-(methylsulfonyl)phenyl)-5(trifluoromethyl)-1H-pyrazol-1-yl)acetamide;

ethyl (4-(4-fluorophenyl)-3-(4-(methylsulfonyl)phenyl)-5-(trifluoromethyl)-1H-pyrazol-1-yl)acetate;

4-(4-fluorophenyl)-3-(4-methylsulfonyl)phenyl)-1-(2-phenylethyl)-1H-pyrazole;

4-(4-fluorophenyl)-3-(4-(methylsulfonyl)phenyl)-1-(2-phenylethyl)-5-(trifluoromethyl)pyrazole;

1-ethyl-4-(4-fluorophenyl)-3-(4-methylsulfonyl)phenyl)-5-(trifluoromethyl)-1H-pyrazole;

5-(4-fluorophenyl)-4-(4-methylsulfonyl)phenyl)-2-(trifluoromethyl)-1H-imidazole;

4-(4-methylsulfonyl)phenyl)-5-(2-thiophenyl)-2-(trifluoromethyl)-1H-imidazole;

5-(4-fluorophenyl)-2-methodoxy-4-(methylsulfonyl)phenyl)-6-(trifluoromethyl)pyridine;

2-ethoxy-5-(4-fluorophenyl)-4-(4-(methylsulfonyl)phenyl)-6-(trifluoromethyl)pyridine;

5-(4-fluorophenyl)-4-(4-(methylsulfonyl)phenyl)-2-(2-propynyloxy)-6-(trifluoromethyl)pyridine;

2-bromo-5-(4-fluorophenyl)-4-(4-methylsulfonyl)phenyl)-6-(trifluoromethyl)pyridine;

4-(2-(3-chloro-4-methoxyphenyl)-4,5-difluorophenyl)benzensulfonamide;

1-(4-fluorophenyl)-2-(4-methylsulfonyl)phenyl)benzene;

5-difluoromethyl-4-(4-methylsulfonyl)phenyl)-3-phenylisoxazole;

4-(3-ethyl-5-phenylisoxazol-4-yl)benzensulfonamid;

4-(5-difluoromethyl-3-phenylisoxazol-4-yl)benzenesulfonamide;

4-(5-hydroxymethyl-3-phenylisoxazol-4-yl)benzenesulfonamide;

4-(5-methyl-3-phenylisoxazol-4-yl)benzenesulfonamide;

1-(2-(4-fluorophenyl)cyclopenten-1-yl)-4-(methylsulfonyl)benzene;

1-(2-(4-fluoro-2-methylphenyl)cyclopenten-1-yl)-4-(methylsulfonyl)benzene;

1-(2-(4-chlorophenyl)cyclopenten-1-yl)-4-(methylsulfonyl)benzene;

1-(2-(2,4-dichlorophenyl)cyclopenten-1-yl)-4-(methylsulfonyl)benzene;

1-(2-(4-trifluoromethylphenyl)cyclopenten-1-yl)-4-(methylsulfonyl)benzene;

1-(2-(4-methylthiophenyl)cyclopenten-1-yl)-4-(methylsulfonyl)benzene;

1-(2-(4-fluorophenyl)-4,4-dimethylcyclopenten-1-yl)-4-(methylsulfonyl)benzene;

4-(2-(4-fluorophenyl)-4,4-dimethylcyclopenten-1-yl)benzesulfonamide;

1-(2-(4-chlorophenyl)-4,4-dimethylcyclopenten-1-yl)-4-(methylsulfonyl)benzene;

4-(2-(4-chlororophenyl)-4,4-dimethylcyclopenten-1-yl)benzenesulfonamide;

4-(2-(4-fluorophenyl)cyclopenten-1-yl)benzenesulfonamide;

4-(2-(4-chlorophenyl)cyclopenten-1-yl)benzenesulfonamide;

1-(2-(4-methoxyphenyl)cyclopenten-1-yl)-4-(methylsulfonyl)benzene;

1-(2-(2,3-difluorophenyl)cyclopenten-1-yl)-4-(methylsulfonyl)benzene;

4-(2-(3-fluoro-4methodyphenyl)cyclopenten-1-yl)benzenesulfonamide;

1-(2-(3-chloro-4-methoxyphenyl)cyclopenten-1-yl)-4-(methylsulfonyl)benzene;

4-(2-(3-chloro-4-fluorophenyl)cyclopenten-1-yl-benzenesulfonamide;

4-(2-(2-methylpyridin-5-yl)cyclopenten-1-yl)benzenesulfonamide;

ethyl 2-(4-(4-fluorophenyl)-5-(4-methylsulfonyl)phenyl)oxazol-2-yl)-2-benzyl-accetate;

2-(4-(4-fluorophenyl)-5-(4-(methylsulfonyl)phenyl)oxazol-2-yl)acetic acid;

2-(tert-butyl)-4-(4-fluorophenyl-5-(4-methylsulfonyl)phenyl)oxazole;

4-(4-fluorophenyl)-5-(4-(methylsulfonyl)phenyl)-2-phenyloxazole;

4-(4-fluorophenyl)-2-methyl-5-(4-methylsulfonyl)phenyl)oxazole; and

4-(5-(3-fluoro-4-methoxyphenyl)-2-trifluoromethyl-4-oxazolyl)benzenesulfonamide;

or a pharmaceutically acceptable salt thereof.

The COX-2 inhibitors used in the pharmaceutical compositions and treatment methods of the present invention are typically those which have an IC50 for COX-2 inhibition less than about 2 μM and an IC50 for COX-1 inhibition greater than about 5 μM, e.g. when measured in the assays described by Brideau et al.in Inflamm. Res. 45:68-74 (1996). Preferably the COX-2 inhibitor has a selectivity ratio of at least 10, more preferably at least 40, for COX-2 inhibition over COX-1 inhibition.

Thus, for example, suitable COX-2 inhibitors for use in the invention may include the following compounds or derivatives thereof or a pharmaceutically acceptable salt thereof, or any hydrate thereof: rofecoxib, etoricoxib, celecoxib, valdecoxib, parecoxib, or a 5-alkyl-2-arylaminophenylacetic acid derivative COX-2 inhibitor, e.g. of formula V as defined below.

In one aspect the present invention relates to pharmaceutical compositions for the treatment or prevention of cardiac and renal related conditions which comprise in combination the AT 1-receptor blocker valsartan or a pharmaceutically acceptable salt thereof and a COX-2 inhibitor of formula V or a pharmaceutically acceptable salt thereof and a pharmaceutically acceptable carrier for simultaneous, sequential or separate use.

In a preferred embodiment this invention to provide a pharmaceutical composition, e.g., for the treatment or prevention of cardiac and renal related condition or disease, ie, selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke which composition comprises (i) the AT 1-receptor blocker valsartan or a pharmaceutically acceptable salt thereof and (ii) a COX-2 inhibitor selected from the group of: compound of formula V, preferably 5-methyl-2-(2′-chloro-6′fluororanilino)phenylacetic acid, 5-methyl-2(2′,4′-difluoro-6′-chloroanilino)phenylacetic acid,5-ethyl-2-(2′,3′,5′,6′-tetrafluoroanilino)phenylacetic acid or a pharmaceutically acceptable salt thereof and a pharmaceutically acceptable carrier for simultaneous, sequential or separate use.

In this composition, components (i) and (ii) can be obtained and administered together, one after the other or separately in one combined unit dose form or in two separate unit dose forms. The unit dose form may also be a fixed combination.

In another preferred embodiment the present invention relates to pharmaceutical compositions for the treatment or prevention of cardiac and renal related conditions which comprise in combination the AT 1-receptor blocker valsartan or a pharmaceutically acceptable salt thereof and the COX-2 inhibitor .5-methyl-2-(2′-chloro-6′fluororanilino)phenylacetic acid or a pharmaceutically acceptable salt thereof and a pharmaceutically acceptable carrier for simultaneous, sequential or separate

In another preferred embodiment the present invention relates to pharmaceutical compositions for the treatment or prevention of cardiac and renal related conditions which comprise in combination the AT 1-receptor blocker valsartan or a pharmaceutically acceptable salt thereof and the COX-2 inhibitor 5-methyl-2(2′,4′-difluoro-6′-chloroanilino)phenylacetic acid or a pharmaceutically acceptable salt thereof and a pharmaceutically acceptable carrier for simultaneous, sequential or separate use. use

In another preferred embodiment the present invention relates to pharmaceutical compositions for the treatment or prevention of cardiac and renal related conditions which comprise in combination the AT 1-receptor blocker valsartan or a pharmaceutically acceptable salt thereof and the COX-2 inhibitor 5-ethyl-2-(2′,3′,5′,6′-tetrafluoroanilino)phenylacetic acid or a pharmaceutically acceptable salt thereof and a pharmaceutically acceptable carrier for simultaneous, sequential or separate use.

Furthermore the invention provides the use of the AT1 receptor blocker valsartan for the preparation of a medicament, for use in combination with a COX-2 inhibitor for treatment of cardiac and renal related conditions.

Furthermore the invention provides the use of the AT 1-receptor blocker valsartan for the preparation of a medicament for use in combination with a COX-2 inhibitor selected from the group of: compound of formula V preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier for the treatment or prevention of cardiac and renal related conditions.

Furthermore the invention provides the use of any preferred pharmaceutical composition according to the invention for the treatment or prevention of cardiac and renal related conditions.

In another embodiment, the invention provides the use of the AT 1-receptor blocker valsartan for the preparation of a medicament for use in combination with a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier wherein the condition is selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

The present invention provides a kit comprising in separate containers in a single package pharmaceutical compositions comprising in one container a pharmaceutical composition comprising a COX-2 inhibitor and in a second container a pharmaceutical composition comprising the AT 1-receptor blocker valsartan.

The kit form is particularly advantageous when the separate components must be administered in different dosage forms (e.g., parenteral valsartan formulation and oral COX-2 inhibitor formulation) or are administered at different dosage intervals.

In yet further aspects the invention provides: a kit comprising in separate containers in a single package pharmaceutical compositions comprising in one container a pharmaceutical composition comprising a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier, and in a second container a pharmaceutical composition comprising the AT 1-antagonist receptor blocker valsartan.

The present invention relates to a package comprising the AT 1-receptor blocker, valsartan together with instructions for use in combination with a COX-2 inhibitor for the treatment or prevention of cardiac and renal related conditions.

In yet further aspects the invention provides a package comprising the AT 1-valsartan receptor blocker together with instructions for use in combination with a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier for the treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

In another embodiment the present invention relates to methods of treating cardiac and renal related conditions by administration of a therapeutically effective amount of any preferred pharmaceutical composition according to the invention comprising an AT1 receptor blocker valsartan plus a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier to a mammal in need thereof.

A further aspect of the present invention is a method for the treatment or prevention of cardiac and renal related condition or disease ,e.g,selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, comprising administering a therapeutically effective amount of combination of (i) the AT 1-receptor blocker valsartan or a pharmaceutically acceptable salt thereof and (ii) a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier to a mammal in need of such treatment.

It has surprisingly been found that the pharmaceutical compositions according to the invention can be used for the treatment of hypertension, primary and secondary pulmonary hypertension, without inducing cardiovascular risks (for example prothrombotic activity, myocardial infarction, unstable angina, cardiac thrombus, ischemic stroke and transient ischemic attacks).

It has surprisingly been found that the pharmaceutical compositions according to the invention can be used for the treatment of heart failure such as (acute and chronic) congestive heart failure without inducing cardiovascular risks (for example prothrombotic activity, myocardial infarction, unstable angina, cardiac thrombus, ischemic stroke and transient ischemic attacks).

It has surprisingly been found that the pharmaceutical compositions according to the invention can be used for the treatment of left ventricular dysfunction and hypertrophic cardiomyopathy without inducing cardiovascular risks (for example prothrombotic activity, myocardial infarction, unstable angina, cardiac thrombus, ischemic stroke and transient ischemic attacks).

It has surprisingly been found that the pharmaceutical compositions according to the invention can be used for the treatment of diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic),angina pectoris, diabetes, secondary aldosteronism, without inducing cardiovascular risks (for example prothrombotic activity, myocardial infarction, unstable angina, cardiac thrombus, ischemic stroke and transient ischemic attacks).

It has surprisingly been found that the pharmaceutical compositions according to the invention can be used for the treatment of renal failure conditions, such as diabetic nephropathy, glomerulonephritis, scleroderma, glomerular sclerosis, proteinuria of primary renal disease, and also renal vascular hypertension, diabetic retinopathy without inducing cardiovascular risks (for example prothrombotic activity, myocardial infarction, unstable angina, cardiac thrombus, ischemic stroke and transient ischemic attacks).

It has surprisingly been found that the pharmaceutical compositions according to the invention can be used for the treatment of vascular disorders, such as migraine, peripheral vascular disease, Raynaud's disease, luminal hyperplasia, cognitive dysfunction (such as Alzheimer's), glaucoma and stroke without inducing cardiovascular risks (for example prothrombotic activity, myocardial infarction, unstable angina, cardiac thrombus, ischemic stroke and transient ischemic attacks).

It has surprisingly been found that, a combination of valsartan and a COX-2 inhibitor achieves greater therapeutic effect (a potentiation) than the administration of valsartan or COX-2 inhibitors alone. The combination surprisingly elicits an increased antihypertensive effect in rodent models of hypertension. The combination also surprisingly ameliorates symptoms and improves mortality rates in animal models of heart failure. Furthermore, the combination surprisingly decreases vascular inflammation and improves cardiac function when given after acute myocardial infarction in rodent models. Surprisingly, inflammatory cellular infiltration is significantly reduced by the combination treatment after the induction of myocardial infarction. Equally surprising is the improvement in vascular nitric oxide availability, reduction in the progression of atherosclerosis and enhancement of plaque stability in animal models of atherosclerosis. The combination also unexpectedly improves renal function in an animal model of renal dysfunction.

Greater efficacy can also be documented as a prolonged duration of action. The duration of action can be monitored as either the time to return to baseline prior to the next dose or as the area under the curve (AUC) and is expressed as the product of the change in blood pressure in millimeters of mercury (change in mmHg) and the duration of the effect (minutes, hours or days).

Further benefits are that lower doses of the individual drugs to be combined according to the present invention can be used to reduce the dosage, for example, that the dosages need not only often be smaller but are also applied less frequently, or can be used to diminish the incidence of side effects. Preferred are low dose combination of valsartan and COX-2inhibitor. The combined administration of valsartan or a pharmaceutically acceptable salt thereof and a COX-2 inhibitor or a pharmaceutically acceptable salt thereof results in a significant response in a greater percentage of treated patients, that is, a greater responder rate results, regardless of the underlying etiology of the condition. This is in accordance with the desires and requirements of the patients to be treated.

It can be shown that combination therapy with valsartan and a COX-2 inhibitor results in a more effective antihypertensive therapy (whether for malignant, essential, reno-vascular, diabetic, isolated systolic, or other secondary type of hypertension) through improved efficacy as well as a greater responder rate. The combination is also useful in the treatment or prevention of heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter or detrimental vascular remodeling. It can further be shown that a valsartan and COX-2 inhibitor therapy proves to be beneficial in the treatment and prevention of myocardial infarction and its sequelae. A valsartan plus COX-2 inhibitor combination is also useful in treating atherosclerosis, angina (whether stable or unstable), and renal insufficiency (diabetic and non-diabetic). Furthermore, combination therapy using valsartan and a COX-2 inhibitor can improve endothelial dysfunction, thereby providing benefit in diseases in which normal endothelial function is disrupted such as heart failure, angina pectoris and diabetes. Furthermore, the combination of the present invention may be used for the treatment or prevention of secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

Thus in the present description the terms “treatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having at least one acid group (for example COOH) can also form salts with bases. Corresponding internal salts may furthermore be formed, if a compound comprises, e.g., both a carboxy and an amino group.

The corresponding active ingredient or a pharmaceutically acceptable salt thereof may also be used in form of a hydrate or include other solvents used for crystallization.

Pharmacologically acceptable salts of the AT1 receptor blocker valsartan and COX-2 inhibitors are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

The Agents of the invention, i.e. the COX-2 inhibitor and the AT1 receptor blocker valsartan, are preferably used in the form of pharmaceutical preparations that contain the relevant therapeutically effective amount of each active ingredient (either separately or in combination) optionally together with or in admixture with inorganic or organic, solid or liquid, pharmaceutically acceptable carriers which are suitable for administration. The Agents of the invention may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping.The unit dose form may also be a fixed combination.

Preferably, the COX-2 pharmaceutical compositions are adapted for oral or parenteral (especially oral) administration. Intravenous and oral, first and foremost oral, administration is considered to be of particular importance. Preferably the COX-2 inhibitor active ingredient is in oral form.

The pharmaceutical compositions according to the invention can be prepared in a manner known per se and are those suitable for enteral, such as oral, rectal, aerosol inhalation or nasal administration, and parenteral such as intravenous or subcutaneous administration, or compositions for transdermal administration (e.g. passive or iontophoretic) to mammals (warm-blooded animals), including man. Such compositions comprise a therapeutically effective amount of the pharmacologically active compound, alone or in combination with one or more pharmaceutically acceptable carriers, especially suitable for enteral or parenteral application. Typical oral formulations include tablets, capsules, syrups, elixirs and suspensions. Typical injectable formulations include solutions and suspensions.

Tablets may be either film coated or enteric coated according to methods known in the art.

Preferred are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 85%, preferably about 1to 70%, of the active ingredient.

The typical pharmaceutically acceptable carriers for use in the formulations described above are exemplified by: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, buffers, preservatives, antioxidants, lubricants, flavoring agents, and the like commonly used in pharmaceutical formulations.

Pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable formulations for topical application, e.g. to the skin and eyes, include aqueous solutions, suspensions, ointments, creams, gels or sprayable formulations, for example, for delivery by aerosol or the like.

For example, the pharmaceutical preparations consist of from about 0.1-90%, preferably of from about 1% to about 80%, of the active compounds. Pharmaceutical preparations for enteral or parenteral administration are, for example, in unit dose forms, such as coated tablets, tablets, capsules or suppositories and also ampoules. These are prepared in a manner which is known per se, for example using conventional mixing, granulation, coating, solubulizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

The dosage of COX-2 inhibitor administered is dependent on the species of warm-blooded animal (mammal), the body weight, age and individual condition, and on the form of administration. In case of COX-2 inhibitors, preferred dosage unit forms are, for example, tablets or capsules comprising, e.g., from about 10 mg to about 1000 mg, preferably from about 50 mg to about 800 mg, even more preferably from about 100 mg to about 400 mg and even more preferably at about400 mg, or about 200 mg, or about 100 mg administered once a day.

COX-2 inhibitor formulations in single dose unit form contain preferably from about 1% to about 90%, and formulations not in single dose unit form contain preferably from about 0.1 to 70%, more preferably from 0.1% to about 65%, of the active ingredient. Single dose unit forms such as capsules, tablets or dragées contain e.g. from about 1 mg to about 1500 mg of the active ingredient.

Valsartan is supplied in the form of suitable dosage unit form, for example, a capsule or tablet, and comprising a therapeutically effective amount, e.g. from about 20 to about 320 mg, of valsartan which may be applied to patients. The application of the active ingredient may occur up to three times a day, starting, e.g., with a daily dose of 20 mg or 40 mg of valsartan, increasing via 80 mg daily and further to 160 mg daily up to 320 mg daily. Preferably, valsartan is applied once a day or twice a day in heart failure patients with a dose of 80 mg or 160 mg, respectively, each. Corresponding doses may be taken, for example, in the morning, at mid-day or in the evening. Preferred is q.d. or b.i.d. administration in heart failure.

The person skilled in the pertinent art is fully enabled to select a relevant test model to prove the efficacy of a combination of the present invention in the hereinbefore and hereinafter indicated therapeutic indications.

For example, representative studies are carried out with a combination of valsartan and COX-2 inhibitor, e.g., applying the following methodology:

Drug efficacy is assessed in various animal models including the spontaneously hypertensive rat (SHR) maintained on a normal, high or low salt diet.

Blood pressure will be monitored in the chronic study procedure by means of a radiotransmitter. The radiotransmitter is surgically implanted into the abdominal aorta of rats. Blood pressure is chronically monitored for periods of up 6 weeks.

Blood pressure, heart rate and activity are determined at various pre-selected time points before, during, and after drug administration. All measurements are performed in unrestrained and undisturbed animals. The maximum study time, determined by battery life, could be as long as nine months. For studies of this duration, rats are dosed orally (1-3 mL/kg vehicle), no more than twice daily or drug is administered via the drinking water or mixed with food. For studies of a longer duration, that is, up to 8 weeks, drugs are given via subcutaneously implanted osmotic minipumps. Osmotic minipumps are selected based on drug delivery rate and time. Valsartan dosages range from 1-50 mg/kg/day.

The hypertensive background of the SHR is modified either by chronic salt loading in an effort to suppress the renin angiotensin system (RAS) or chronic salt depletion to activate the RAS in the SHR. These manipulations will be carried out to more extensively evaluate the efficacy of the various test substances. Experiments performed in SHR are supplied by Taconic Farms, Germantown, N.Y. (Tac:N(SHR)fBR). A radiotelemetric device (Data Sciences International, Inc., St. Paul, Minn.) is implanted into the lower abdominal aorta of all test animals between the ages of 14 to 16 weeks of age. All SHR are allowed to recover from the surgical implantation procedure for at least 2 weeks prior to the initiation of the experiments. Cardiovascular parameters are continuously monitored via the radiotransmitter and transmitted to a receiver where the digitized signal is then collected and stored using a computerized data acquisition system. Blood pressure (mean arterial, systolic and diastolic pressure) and heart rate are monitored in conscious, freely moving and undisturbed SHR in their home cages. The arterial blood pressure and heart rate are measured every 10 minutes for 10 seconds and recorded. Data reported for each rat represent the mean values averaged over a 24-hour period and are made up of the 144-10 minute samples collected each day. The baseline values for blood pressure and heart rate consist of the average of three consecutive 24-hour readings taken prior to initiating the drug treatments. All rats are individually housed in a temperature and humidity controlled room and are maintained on a 12-hour light dark cycle.

In addition to the cardiovascular parameters, weekly determinations of body weight also are recorded in all rats. Treatments are administered in the drinking water, via daily oral gavage or in osmotic minipumps as stated above. If given in drinking water, water consumption is measured five times per week. Valsartan and COX-2 inhibitor doses for individual rats are then calculated based on water consumption for each rat, the concentration of drug substance in the drinking water, and individual body weights. All drug solutions in the drinking water are made up fresh every three to four days. Typical dosages for valsartan in drinking water range from 1-50 mg/kg/day whereas the dosage of COX-2 inhibitor is highly dependent upon the specific agent used. In most situations, a daily dose will not exceed 20 mg/kg/day when administered as the monotherapy. In combination, lower dosages of each agent are used and correspondingly, valsartan is given in the range of 1-30 mg/kg/day and COX-2 inhibitor in dosages below 1 mg/kg/day. However, in cases wherein the responder rate is increased with combination treatment, the dosages are identical to those used as monotherapy.

When drugs are administered by oral gavage, the dose of valsartan ranges from 1-50 mg/kg/day and COX-2 inhibitor does not exceed 0.5-20 mg/kg/day.

Upon completion of the chronic studies, rats are anesthetized and the heart rapidly removed. After separation and removal of the atrial appendages, left ventricle and left plus right ventricle (total) are weighed and recorded. Left ventricular and total ventricular mass are then normalized to body weight and reported. All values reported for blood pressure and cardiac mass represent the group mean±sem.

Vascular function and structure are evaluated after treatment to assess the beneficial effects of the combination. SHR are studied according to the methods described by Intengan et al., Circulation, Vol. 100, No. 22, pp. 2267-2275 (1999).

Apolipoprotein E-deficient (ApoE−/−), LDLR−/−, C57BL/6 mice and COX-2± heterozygous mice (Jackson Laboratories, Maine) can be used for demonstrating the beneficial effects of the combination of valsartan and cox-2 inhibitors on the progression of atherosclerosis. The animals are maintained on a diet containing 21% fat and 0.15% cholesterol (Teklad) and treated with a combination of valsartan and the cox-2 inhibitor. Treatment with the drugs are initiated when the mice are 9-10 weeks old. Serum cholesterol, triglycerides and urinary prostaglandin metabolites are measured. Arterial lesions are quantitated using aortic segments obtained from the aortic arch. Cryosections taken from the aorta are stained with oil-red O and counterstained with hematoxylin, and quantitative analysis of lipid-stained lesions was performed using an imaging system KS 300 (Release 2.0, Kontron Electronik GmbH).20 Sections were also stained for collagen content with Masson's trichrome. To detect macrophages in the arterial lesions, cryosections of the aorta are incubated with monoclonal rat antibody to mouse macrophages, MOMA-2.

Myocardial infarction (MI) is produced in Sprague-Dawley rats by ligation of the left coronary artery. At four weeks after surgery, animals are treated with the combination of valsartan and cox-2 inhibitor. At 16 weeks after surgery, the animals are examined for hemodynamic function, euthanized and the hearts weighed. For assessments of hemodynamic function, rats are anesthetized with sodium pentobarbital (50 mg/kg i.p.). A miniature pressure transducer catheter (Millar Micro-Tip) is inserted into the right carotid artery and then advanced into the left ventricle. Left ventricular end-diastolic and left ventricular peak systolic pressures are recorded. After these assessments, the rats are sacrificed and the heart excised for weighing.

The available results indicate an unexpected therapeutic effect of a combination according to the invention.

The following examples illustrate the above-described invention; however, it is not intended to restrict the scope of this invention in any manner.

### EXAMPLES

### A. COX-2 Inhibitor Formulation Examples

### Example 1

TABLE 1

| | Amount per 200 mg |
|---|---|
| Ingredient | tablet batch (kg) |
| Core | |
| Granulation | |
| 5-methyl-2-(2′-chloro-6′- | 50** |
| fluoroanilino)phenylacetic acid drug | |
| substance | |
| Microcrystalline cellulose, NF (PH | 12.85 |
| 101) | |
| Lactose monohydrate, NF | 11.65 |
| Croscarmellose sodium, NF | 1 |
| Povidone, USP | 4 |
| Titanium dioxide, USP | 2 |
| Water, purified***, USP | 20.375 |
| Extra-granular Phase | |
| Microcrystalline cellulose, NF (PH | 13 |
| 102) | |
| Croscarmellose sodium, NF | 3 |
| Titanium dioxide, USP | 2 |
| Magnesium stearate, NF | 0.5 |
| Coating | |
| Opadry white | 2.801**** |
| Opadry yellow | 2.0**** |
| Opadry red | 0.4**** |
| Opadry black | 0.0504**** |
| Water, purified***, USP | 29.758**** |

**The weight of drug substance is taken with reference to the dried substance (100 per cent) on the basis of the assay value (factorization). The difference in weight is adjusted by the amount of microcrystalline cellulose used.
***Removed during processing.
****Includes a 50% excess for loss during the coating process.

Table 1, above, sets out the formula for a batch of approximately 250,000 immediate release film-coated tablets of 5-methyl-2-(2′-chloro-6′-fluoroanilino)-phenylacetic acid. To make the tablets, titanium dioxide is dispersed in water, followed by the addition of povidone and mixing for 20 minutes to make a povidone/titanium dioxide suspension. The drug substance, lactose, microcrystalline cellulose, and croscarmellose are mixed in a high shear mixer (e.g., a Collette Gral) for 5 minutes to form a drug mixture. The drug mixture is granulated in the high shear mixer with the povidone/titanium dioxide suspension. The suspension is pumped at a rate of 3 kg/min into the drug mixture. The resulting mixture is mixed an additional 90 seconds after all the suspension is added. The wet granulation is dried in a fluid bed dryer, using an inlet air temperature of 50° C. The residual water target is 3.5% (with a permissible range of 2.5-4.5%). The dried granulation is passed through a screen using a mill (oscillator) and a 30 mesh screen. The previous steps are repeated to make a second granulation.

The extra-granular phase titanium dioxide is passed through a 60 mesh hand screen. The dry granulations are mixed with the extra-granular phase microcrystalline cellulose, croscarmellose sodium and titanium dioxide in a twin shell mixer for 300 revolutions to form a penultimate mixture. Magnesium stearate is passed through a 60 mesh hand screen and is mixed with the penultimate mixture in a twin shell mixer for 50 revolutions to form a tableting mixture. The tableting mixture is pressed into tablets using a tablet press and oval punches.

The coating powders (Opadry) are mixed with purified water to make a 15% w/w coating suspension. The tablets are film coated with the coating suspension in a coating pan using 60° C. to 75° C. inlet air temperature.

Table 2 sets out the contents of a 200 mg 5-methyl-2-(2′-chloro-6′-fluoroanilino)phenylacetic acid film-coated tablet.

TABLE 2

| | Theoretical | |
|---|---|---|
| Ingredient | amount [mg] | Function |
| Core | | |
| 5-methyl-2-(2′-chloro-6′- | 200 | Active |
| fluoroanilino)phenylacetic acid | | substance |
| drug substance | | |
| Microcrystalline cellulose (PH | 51.4 | Filler |
| 101) | | |
| Lactose | 46.6 | Filler |
| Povidone | 16 | Binder |
| Titanium dioxide | 8 | Color |
| Croscarmellose sodium | 4 | Disintegrant |
| Water, purified* | Q.S. | Granulating |
| | | liquid |
| Extragranular phase | | |
| Microcrystalline cellulose (PH | 52 | Filler |
| 102) | | |
| Croscarmellose sodium | 12 | Disintegrant |
| Titanium dioxide | 8 | Color |
| Magnesium stearate | 2 | Lubricant |
| Core weight | 400 | |
| Coating | | |
| Opadry white (00F18296) | 7.4676 | Color |
| Opadry yellow (00F12951) | 5.3312 | Color |
| Opadry red (00F15613) | 1.0668 | Color |
| Opadry black (00F17713) | 0.1344 | Color |
| Water, purified* | Q.S. | Coating solvent |
| Total weight | 414 | |

*removed during processing

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1

### Example 2

An alternative formulation is as set out in Table 3, with information about as percentage w/w, mg/dose, and kg/50,000 tablet batch.

TABLE 3
(a) Alternative formulation composition

| | | | |
|---|---|---|---|
| % w/w | Ingredient | Mg/dose | Kg/batch |

Granulation

| | | | |
|---|---|---|---|
| 65.04 | 5-methyl-2-(2′-Chloro-6′-fluoroanilino) | 400.00 | 20.00 |
| | phenylacetic acid drug substance | | |
| 2.15 | Croscarmellose sodium, NF (Ac-Di-Sol) | 13.22 | 0.661 |
| 6.60 | Povidone K30, USP | 40.59 | 2.029 |
| 18.12 | Purified water, USP* | Qs | Qs |

Blending

| | | | |
|---|---|---|---|
| 23.56 | Microcrystalline Cellulose, NF (Avicel | 144.90 | 6.066 |
| | PH 102) | | |
| 2.15 | Croscarmellose sodium, NF (Ac-Di-Sol) | 13.22 | 0.553 |
| 0.50 | Magnesium Stearate, NF (vegetable | 3.07 | 0.128 |
| | source) | | |

Film Coating

| | | | |
|---|---|---|---|
| 84.46 | Opadry, Global White 00F18296 | 15.2028 | 0.296637 |
| 14.03 | Opadry, Global Red 00F15613 | 2.5254 | 0.049275 |
| 1.51 | Opadry, Global Black 00F17713 | 0.2718 | 0.005303 |
| | Purified Water, USP* | Qs | 1.990218 |
| | Film Coated Tablet Weight | 633.00 | |

*Does not appear in final product. Percentage of water added used for granulation based on the dry weight of drug substance and croscarmellose sodium.

The batch is granulated as described in Example 1. The granulation is dried to residual moisture (% LOD) of 1.79%. The formulation process is the same as for the development batches as described above, except for the additional step of coating with Opadry in a coating pan. The coating powders (Opadry) are mixed with purified water to make a 15% w/w coating suspension. The tablets are film coated with the coating suspension in a coating pan using 60° C. to 75° C. inlet air temperature. Based on friability data, a target force of 18 KN (16-20 KN range) is used to compress the remainder of the batch, resulting in acceptable friability (less than 0.5%) and the disintegration times of less than 5 mins. The ejection force is approximately 800 N throughout the compression run. This demonstrates that the blend is lubricated adequately. No picking/sticking is observed on the punch surfaces after 225 minutes. Thus, a smaller size tablet with high drug loading (65%) is achieved using a high shear granulation process, using 17×6.7 mm ovaloid tooling to get tablets with acceptable hardness and friability characteristics.

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1%.

### Example 3

Wet granulated tablet composition

| Amount per tablet | Ingredient |
|---|---|
| 25 mg | COX-2 inhibitor |
| 79.7 mg | Microcrystalline cellulose |
| 79.7 mg | Lactose monohydrate |
| 6 mg | Hydroxypropyl cellulose |
| 8 mg | Croscarmellose sodium |
| 0.6 mg | Iron oxide |
| 1 mg | Magnesium stearate |

Tablet dose strengths of between 5 and 125 mg can be accomodated by varying total weight, and the ratio of the first three ingredients. Generally it is preferable to maintain a 1:1 ratio for microcrystalline cellulose: lactose monohydrate.

### Example 4

Directly compressed tablet composition

| Amount per tablet | Ingredient |
|---|---|
| 25 mg | COX-2 inhibitor |
| 106.9 mg | Microcrystalline cellulose |
| 106.9 mg | Lactose anhydrate |
| 7.5 mg | Croscarmellose sodium |
| 3.7 mg | Magnesium stearate |

Tablet dose strengths of between 5 and 125 mg can be accomodated by varying total tablet weight, and the ratio of the first three ingredients. Generally it is preferable to maintain a 1:1 ratio for microcrystalline cellulose:lactose monohydrate.

### Example 5

Hard gelatine capsule composition

| Amount | | |
|---|---|---|
| per capsule | | Ingredient |
| 25 | mg | COX-2 inhibitor |
| 37 | mg | Microcrystalline cellulose |
| 37 | mg | Lactose anhydrate |
| 1 | mg | Magnesium stearate |
| 1 | capsule | Hard gelatin capsule |

Capsule dose strengths of between 1 and 50 mg can be accomodated by varying total fill weight, and the ratio of the first three ingredients. Generally it is preferable to maintain a 1:1 ratio for microcrystalline cellulose:lactose monohydrate.

### Example 6

Oral solution

| Amount | | |
|---|---|---|
| per | | |
| 5 mL | | Ingredient |
| 50 | mg | COX-2 inhibitor |
| to 5 | mL | with Polyethylene oxide 400 |

### Example 7

Oral suspension

| Amount | | |
|---|---|---|
| per 5 mL | | |
| dose | | Ingredient |
| 101 | mg | COX-2 inhibitor |
| 150 | mg | Polyvinylpyrrolidone |
| 2.5 | mg | Poly oxyethylene sorbitan monolaurate |
| 10 | mg | Benzoic acid |
| to 5 | mL | with sorbitol solution (70%) |

Suspension dose strengths of between 1 and 50 mg/5 ml can be accomodated by varying the ratio of the first two ingredients.

### Example 8

Intravenous infusion

| Amount per | | |
|---|---|---|
| 200 mL dose | | Ingredient |
| 1 | mg | COX-2 inhibitor |
| 0.2 | mg | Polyethylene oxide 400 |
| 1.8 | mg | Sodium chloride |
| to 200 | mL | Purified water |

### B) Examples of Valsartan Formulation

### Formulation Example 1

Film-Coated Tablets:

| | | |
|---|---|---|
| | Composition | |
| Components | Per Unit (mg) | Standards |

Granulation

| | | |
|---|---|---|
| Valsartan [= active ingredient] | 80.00 | |
| Microcrystalline cellulose/ | 54.00 | NF, Ph. Eur |
| Avicel PH 102 | | |
| Crospovidone | 20.00 | NF, Ph. Eur |
| Colloidal anhydrous silica/colloidal | 0.75 | Ph. Eur/NF |
| silicon dioxide/Aerosil 200 | | |
| Magnesium stearate | 2.5 | NF, Ph. Eur |

Blending

| | | |
|---|---|---|
| Colloidal anhydrous silica/colloidal | 0.75 | Ph. Eur/NF |
| silicon dioxide/Aerosil 200 | | |
| Magnesium stearate | 2.00 | NF, Ph. Eur |

Coating

| | |
|---|---|
| Purified water*) | — |
| DIOLACK pale red 00F34899 | 7.00 |
| TOTAL TABLET MASS | 167.00 |

*)Removed during processing.

The film-coated tablet is manufactured, e.g., as follows:

A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screening mill. The resulting mixture is again pre-mixed in a diffusion mixer, compacted in a roller compactor and then sieve through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tablets are coated with a film by using Diolack pale red in a perforated pan.

### Formulation Example 2

Film-Coated Tablets:

| | | |
|---|---|---|
| | Composition | |
| Components | Per Unit (mg) | Standards |

Granulation

| | | |
|---|---|---|
| Valsartan [=active ingredient] | 160.00 | |
| Microcrystalline cellulose/ | 108.00 | NF, Ph. Eur |
| Avicel PH 102 | | |
| Crospovidone | 40.00 | NF, Ph. Eur |
| Colloidal anhydrous silica/colloidal | 1.50 | Ph. Eur/NF |
| silicon dioxide/Aerosil 200 | | |
| Magnesium stearate | 5.00 | NF, Ph. Eur |

Blending

| | | |
|---|---|---|
| Colloidal anhydrous silica/colloidal | 1.50 | Ph. Eur/NF |
| silicon dioxide/Aerosil 200 | | |
| Magnesium stearate | 4.00 | NF, Ph. Eur |

Coating

| | |
|---|---|
| Opadry Light Brown 00F33172 | 10.00 |
| TOTAL TABLET MASS | 330.00 |

The film-coated tablet is manufactured, e.g., as described in Formulation Example 1.

### Formulation Example 3

Film-Coated Tablets:

| | | |
|---|---|---|
| | Composition | |
| Components | Per Unit (mg) | Standards |

Core: Internal phase

| | | |
|---|---|---|
| Valsartan [=active ingredient] | 40.00 | |
| Silica, colloidal anhydrous (Colloidal | 1.00 | Ph. Eur, USP/NF |
| silicon dioxide) [=Glidant] | | |
| Magnesium stearate [=Lubricant] | 2.00 | USP/NF |
| Crospovidone [Disintegrant] | 20.00 | Ph. Eur |
| Microcrystalline cellulose [=Binding | 124.00 | USP/NF |
| agent] | | |

External phase

| | | |
|---|---|---|
| Silica, colloidal anhydrous, (Colloidal | 1.00 | Ph. Eur, USP/NF |
| silicon dioxide) [=Glidant] | | |
| Magnesium stearate [Lubricant] | 2.00 | USP/NF |

Film coating

| | |
|---|---|
| Opadry ® brown OOF 16711*) | 9.40 |
| Purified Water**) | — |
| TOTAL TABLET MASS | 199.44 |

*)The composition of the Opadry ® brown OOF16711 coloring agent is tabulated below.
**)Removed during processing

Opadry® Composition:

| | Approximate % |
|---|---|
| Ingredient | Composition |
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablet is manufactured, e.g., as described in Formulation Example 1.

### Formulation Example 4

Capsules:

| | Composition |
|---|---|
| Components | Per Unit (mg) |
| Valsartan [=active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |

Shell

| | |
|---|---|
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| TOTAL TABLET MASS | 209.50 |

The tablet is manufactured, e.g., as follows:

Granulation/Drying

Valsartan and microcrystallin cellulose are spray-granulated in a fluidized bed granulator with a granulating solution consisting of povidone and sodium lauryl sulphate dissolved in purified water. The granulate obtained is dried in a fluidized bed dryer.

Milling/Blending

The dried granulate is milled together with crospovidone and magnesium stearate. The mass is then blended in a conical srew type mixer for approximately 10 minutes.

Encapsulation

The empty hard gelatin capsules are filled with the blended bulk granules under controlled temperature and humidity conditions. The filed capsules are dedusted, visually inspected, weightchecked and quarantined until by Quality assurance department.

### Formulation Example 5

Capsules:

| | Composition |
|---|---|
| Components | Per Unit (mg) |
| Valsartan [=active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |

Shell

| | |
|---|---|
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| TOTAL TABLET MASS | 342.00 |

The formulation is manufactured, e.g., as described in Formulation Example 4.

### Formulation Example 6

Hard Gelatine Capsule:

| Components | Composition Per Unit (mg) |
|---|---|
| Valsartan [= active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| TOTAL TABLET MASS | 130.00 |

### Formulation Example 7

A hard gelatin capsule, comprising as active ingredient, e.g., (S)-N-(1-carboxy-2-methylprop-1-yl)-N-pentanoyl-N-[2′(1H-tetrazol-5-yl)biphenyl-4-yl-methyl]amine, can be formulated, for example, as follows:

Composition:

| | |
|---|---|
| (1) valsartan | 80.0 mg |
| (2) microcrystalline cellulose | 110.0 mg |
| (3) polyvidone K30 | 45.2 mg |
| (4) sodium lauryl sulfate | 1.2 mg |
| (5) crospovidone | 26.0 mg |
| (6) magnesium stearate | 2.6 mg |

Components (1) and (2) are granulated with a solution of components (3) and (4) in water. The components (5) and (6) are added to the dry granulate and the mixture is filled into size 1 hard gelatin capsules.

All publications and patents mentioned herein are incorporate by reference in their entirety as if set forth in full herein.

What is claimed is:

1. A pharmaceutical composition comprising (i) the AT 1-receptor blocker valsartan or a pharmaceutically acceptable salt thereof and (ii) a COX-2 inhibitor selected from the group of: compound of formula V 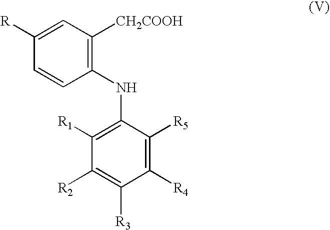
wherein R is methyl or ethyl;
R1 is chloro or fluoro;
R2 is hydrogen or fluoro;
R3 is hydrogen, fluoro, chloro, methyl, ethyl, methoxy, ethoxy or hydroxy;
R4 is hydrogen or fluoro; and
R5 is chloro, fluoro, trifluoromethyl or methyl; preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier.

2. A composition according to claim 1 in which the COX-2 inhibitor is a COX-2 inhibitor which has an IC50 for COX-2 inhibition less than about 2 μM and an IC50 for COX-1 inhibition greater than about 5 μM.

3. A composition for the treatment or prevention of cardiac and renal related conditions which comprises in combination the AT 1-receptor blocker valsartan and a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier for simultaneous, sequential or separate use.

4. A composition according to claim 3 wherein the condition is selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

5. Use of the AT 1-receptor blocker valsartan for the preparation of a medicament for use in combination with a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier for the treatment or prevention of cardiac and renal related conditions.

6. Use according to claim 5 wherein the condition is selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

7. A package comprising the AT 1-receptor blocker valsartan together with instructions for use in combination with a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier for the treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

8. A kit comprising in separate containers in a single package pharmaceutical compositions comprising in one container a pharmaceutical composition comprising a COX-2 inhibitor selected from the group of: compound of formula V, preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier, and in a second container a pharmaceutical composition comprising the AT 1-antagonist receptor blocker valsartan.

9. A method for the treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, comprising administering a therapeutically effective amount of combination of (i) the AT 1-antagonists receptor blocker valsartan or a pharmaceutically acceptable salt thereof and (ii) a COX-2 inhibitor selected from the group of: compound of formula V preferably 5-methyl-2-(2′-chloro-6′fluororanilino)phenylacetic acid,5-methyl-2(2′,4′-difluoro-6′-chloroanilino)phenylacetic acid, 5-ethyl-2-(2′,3′,5′,6′-tetrafluoroanilino)phenylacetic acid or a pharmaceutically acceptable salt thereof and a pharmaceutically acceptable carrier to a mammal in need of such treatment.
